SEQUENCE LISTING (8 sequences)

SEQ ID NO: 1 - DNA, 531 bp, Penaeus monodon
  Nucleotide sequence of insulin-like androgenic gland peptides (IAG)
atgaaccaactcgctgcctcacgcacctacggcctcggcatggccactcagctgcttgtgggaatgctgatggtcctctccctgacttcggcgtcaagctgctacaacgtcacggggattcctgttgacttcgactgcggtgacatcggcgataccatgagtcacatctgcaagacgtttcccacagcccggcctcactcgcgaatgccttctctttcccttacagtgtccaggtcagctgataccgacgacctctggcaggacacgagagcaggtcagaccacgcccattgacctgcttcctcgccagtaccgcctgcatccccgggccttgaatccgatgcgatatttcgaaatggacctgataagagaccacctggtgagccccgaagccgcgcacgccctcgtcaagacatccaggaggcgcgcgaagagatcctacaacgtgcaggatgagtgctgcaaccacgtgagccagcggacgtgtgtggcggaggagattctggagtattgcgaggacccgtacttctaa

SEQ ID NO: 2 - DNA, 339 bp, Litopenaeus vannamei
  Nucleotide sequence of insulin-like androgenic gland peptides (IAG)
atctgcaagacgtttcccacggccaggccccacgtgagagtgtcaaggtcagccgataccgacgacctctggcaggacgcgggggcaggtcagacaacgccccctgacctgctcccccgccggcaccgcctccaccccagggccctgaatccaacgtggaatctcgaaagggacctgatcagagacatcctagtgagccccgaagccgcgcacgccctcgtcaggacgccccggagccgcgcgaagaggtcctacaacgtgcaggacgagtgctgcaaccacgtgagccagcggatgtgtgtggcggaggagatcctggagtattgcgaggacccgtac

SEQ ID NO: 3 - DNA, 500 bp, Fenneropenaeus chinensis
  Nucleotide sequence of insulin-like androgenic gland peptides (IAG)
atgatccctctcactgcctcgcgcccccacggcctcggcatacccactcaactgctcgtgggcgtgctggtggtcctttccctgacttcgacgtcgagtggctacaacgtcaccgggattcctgttgacttcgactgcggggacatcggcgacaccatgagtcagatctgcaagacgttccccacggccaggccttacgcgcgagtgtcaaggtcagctgataccgacgacctctggcatgacacgggagcagatcagaccacgccccttgacctgctcagtcgccagtatcgcctccactgaatccgatgcgatatctcgaaagggacctgacaaagcacatcctggtgagccgcgaagccgcgcacgccctcgtcaagacatccgggagtcgcgtgaagagatcctacaacgtgcaggacgagtgctgcaaccacgtgagccagcggacgtgtgtggcggaggagattctggagtattgcgaggacccggtaccttag

SEQ ID NO: 4 - protein, 176 aa, Penaeus monodon
  Amino acid sequence of insulin-like androgenic gland peptides (IAG)
MNQLAASRTYGLGMATQLLVGMLMVLSLTSASSCYNVTGIPVDFDCGDIGDTMSHICKTFPTARPHSRMPSLSLTVSRSADTDDLWQDTRAGQTTPIDLLPRQYRLHPRALNPMRYFEMDLIRDHLVSPEAAHALVKTSRRRAKRSYNVQDECCNHVSQRTCVAEEILEYCEDPYF

SEQ ID NO: 5 - protein, 113 aa, Litopenaeus vannamei
  Amino acid sequence of insulin-like androgenic gland peptides (IAG)
ICKTFPTARPHVRVSRSADTDDLWQDAGAGQTTPPDLLPRRHRLHPRALNPTWNLERDLIRDILVSPEAAHALVRTPRSRAKRSYNVQDECCNHVSQRMCVAEEILEYCEDPY

SEQ ID NO: 6 - protein, 169 aa, Fenneropenaeus chinensis
  Amino acid sequence of insulin-like androgenic gland peptides (IAG)
MIPLTASRPHGLGIPTQLLVGVLVVLSLTSTSSGYNVTGIPVDFDCGDIGDTMSQICKTFPTARPYARVSRSADTDDLWHDTGADQTTPLDLLSRQYRLHPRALNPMRYLERDLTKHILVSREAAHALVKTSGSRVKRSYNVQDECCNHVSQRTCVAEEILEYCEDPVP

SEQ ID NO: 7 - protein, 173 aa, Macrobrachium rosenbergii
  Amino acid sequence of insulin-like androgenic gland peptides (IAG)
MGYWNAEIKCVLFCSLVASLLPQPSSSYEIECLSVDFDCGDITNTLASVCLRHNNYINPGPTYVSKERRSADIYTVPSTKSPSLAHPRATHLTMADEETQKVSKVEEEIQHMTLSREEANNMLHSKRRFRRDSVRRSPREECCNNASFRRCNFEEVAEYCIELRPGVNTCSSR

SEQ ID NO: 8 - protein, 154 aa, Jasus edwardsii
  Amino acid sequence of insulin-like androgenic gland peptides (IAG)
MLAPILLKLVVIAGMRPRPVVSYRVEGLSVDFQCGELEDSINSICAEHREKRSVNTAAVGASRDGGTDPSRRPYRHPRAIQMVLSHPTTLAGAVQQGAGAVEGVRVTRQAALSYVKSRIQDTNETNLQDECCPVPLVRHCEMEEILEYCYLMEG

(19) United States
(12) Patent Application Publication
(10) Pub. No.: US 2017/0035034 A1
(43) Pub. Date: Feb. 9, 2017

(54) MODERATION OF CRUSTACEAN ANDROGENIC GLAND PEPTIDE FOR MONO SEX CULTURE

(71) Applicant: Siu Ming Chan, Zhanjiang (CN)

(72) Inventor: Siu Ming Chan, Zhanjiang (CN)

(21) Appl. No.: 15/236,356

(22) Filed: Aug. 12, 2016

Related U.S. Application Data
(63) Continuation of application No. 14/506,690, filed on Oct. 6, 2014, now abandoned.
(60) Provisional application No. 61/887,317, filed on Oct. 4, 2013.

(51) Int. Cl. A01K 61/00; C12N 15/113

(52) U.S. Cl. CPC A01K 61/005; C12N 15/1136; A01K 61/001; A61D 19/027

(57) ABSTRACT

This invention is directed to methods for manipulating sex, reversing sex, changing sex ratio, or moderating crustacean androgenic gland peptide expression in crustaceans for producing a monosex culture. In one embodiment, the present method comprises the step of injecting a dsRNA for the gene of IAG into a male crustacean. In another embodiment, the present method comprises the step of injecting a dsRNA for the gene of IAG into a female crustacean that had acquired a spermatophore (a sperm sac) after mating with a male. In another embodiment, the present invention provides a method of producing a monosex culture of crustacean by introducing a recombinant IAG peptide into the female crustacean, thereby obtains male with altered sexual characteristics for mating with a normal female. In one embodiment, the crustacean is a shrimp or lobster.

### CROSS-REFERENCE TO RELATED APPLICATION

This application claims priority of U.S. Ser. No. 61/887,317, filed Oct. 4, 2014. The content of the preceding application is hereby incorporated in its entirety by reference into this application.

### FIELD OF THE INVENTION

The present invention relates to methods for moderating crustacean androgenic gland peptide for producing a monosex culture.

### BACKGROUND OF THE INVENTION

Most cultured crustacean species exhibit sexually dimorphic growth patterns, that is, one sex grows larger than the other sex. For instance, in two most economically important penaeid shrimps, Litopenaeus vannamei and Penaeus monodon, females grow larger than males. On the contrary, several cultured species such as the Australian red-claw crayfish Cherax quadricarinatus and giant freshwater prawn Macrobrachium rosenbergii, males grow faster and reach heavier weights than females [1]. The culture of monosex populations (either all-male or all-female) is a preferred approach for gaining higher yields, with the ecological benefit of reducing the risk of invasion by the cultured species [2].

It is also suggested that sexual manipulations may also render sustainable solutions to the environmental problems caused by the presence of invasive crustacean species with detrimental impacts ranging from aggressive competition with native species for food and shelter, to affecting aquaculture facilities and harvests and causing structural damage to river banks [2].

Sexual differentiation in crustaceans is regulated specifically by a nonsteroidogenic male-specific androgenic gland (AG) which can induce a sexual shift through steroid administration [3]. A lot of studies have proved that AG implantation could masculinize females and its removal could feminize males in amphipod, isopod and decapod species [2]. AG was detected in all studied malacostracan crustacean species, its key role in male make it a viable target for sexual manipulation-based biotechnologies [2]. Attempt was made to obtain a monosex culture by microsurgical removal of the AG from juvenile males [2, 5]. However, this biotechnology is hampered by the low success rate of the microsurgery and by lengthy and labor-intensive progeny testing [2, 5].

Recently, insulin-like androgenic gland peptides (IAG) were identified in many crustacean species. The first decapod insulin-like AG-specific peptide encoding cDNA (IAG) was discovered in 2007 and IAGs of nine additional decapod species have been cloned [2, 4]. It was reported that IAG silencing by RNA interference (RNAi) in such decapod species was useful in obtaining a full and functional sex reversal, leading to the production of monosex populations that are the progeny of sexually reversed males (‘neo-females’) [2]. ‘Neo-females’ are genotypic males having female sexual characteristics. IAG silencing allows one to obtain monosex populations without any genetic modification. The intervention is temporal and not transmissible to next generations [2].

Sex reversal can be obtained by either knocking down the IAG gene in males to obtain neo-females, or administering IAG gene to females to obtain neo-males. Four biotechnological routes to achieve monosex populations have been suggested [2]. In species where the males are homogametic (i.e., bear two homologous sex chromosomes, ZZ), knocking down the IAG in males to induce neo-females will enable the production of 100% male populations [6, 7]. IAG knockdown or administration can also be done in heterogametic moiety. However, knocking down the IAG gene in XY males would result in YY males which are not 100% viable. On the contrary, a 100% female population can be achieved by inserting IAG gene to the XY females, which is a desirable approach for crabs [8, 2].

The enhanced yield achievable through monosex population culture has been shown in case studies in a crab species [8], in a crayfish species [9, 10] and in a prawn species [11]. Therefore, attaining monosex population culture of crustacean by moderating IAG gene would be very useful and valuable with respect to both the industry and the environment.

In case where obtaining a full male population is desirable, one may employ RNAi knockdown of IAG gene to produce the ‘neo-females’ which are then crossed with normal homogametic male to produce a 100% male (ZZ) population. However, the procedure of RNAi knockdown is highly complicated.

A common approach for RNAi knockdown is to inject dsRNA for IAG gene into the shrimp and differentiate the male and female shrimp afterwards (as dsRNA may not guarantee a 100% knockdown of the gene, some males remain both genotypically and phenotypically male). However, it is observed that RNAi knockdown is not always effective in inducing sex reversal, as observed in some studies the low sex reversal rate or even absence of sex reversal. Furthermore, even if IAG gene has been successfully knockdown, it may not be easy to differentiate their sex due to their small size. Furthermore, this approach usually involves crosses of two generations to obtain 100% monosex population and only a few numbers of “neo-females” can be obtained for the second crossing with normal male.

Another approach to create a monosex culture is to convert normal female to neomale using recombinant or purified IAG protein. The obtained neomale is then crossed with normal female to give a 100% female population. However, as in the RNAi method, sex reversal rate is not always high enough to produce sufficient amount of neomale for subsequent crossing.

The present invention provides an alternative approach for obtaining a 100% monosex population. The methods described herein improved the current methods by injecting the dsRNA for IAG gene or recombinant IAG protein at an appropriate molt stage of the crustacean at which the crustacean is more responsive to the treatment. Furthermore, the methods described herein only require one single cross of the animal and thereby reduce the trouble of differentiating the sex of the animal after treatment of the dsRNA for IAG.

### SUMMARY OF THE INVENTION

The present invention provides a nucleic acid molecule and method for moderating crustacean androgenic gland peptide expression for producing a monosex culture.

In one embodiment, the present invention provides a nucleic acid molecule which is a double-stranded RNA (dsRNA) corresponding to the DNA sequence of insulin-like androgenic gland peptides (IAG) of crustaceans. In another embodiment, the dsRNA described herein is a dsRNA corresponding to the DNA sequence of the insulin-like androgenic gland peptides (IAG) of shrimp or lobster.

In one embodiment, the present invention provides a method of manipulating sex, reversing sex or changing sex ratio in crustaceans by moderating the expression of androgenic gland peptide in the crustaceans. In one embodiment, the expression of androgenic gland peptide is moderated by injecting a dsRNA for the gene of IAG peptide into the crustaceans, thereby induces silencing of the IAG gene in the crustaceans and alters the sexual characteristics of the crustaceans.

In one embodiment, the present invention provides a method of producing a monosex culture of female crustacean comprising the step of injecting a dsRNA corresponding to the DNA sequence of the IAG peptide into a male crustacean. In one embodiment, the dsRNA to be injected is a dsRNA corresponding to the DNA sequence of the IAG peptide of a shrimp or lobster, thereby producing a monosex culture of female shrimp or lobster.

In one embodiment, the present invention provides a method of producing a monosex culture of female crustacean comprising the step of injecting a dsRNA for the gene of IAG into a female crustacean that had acquired a spermatophore (a sperm sac) after mating with a male.

In one embodiment, the present invention provides a method of manipulating sex, reversing sex or changing sex ratio in crustaceans by introducing a recombinant IAG peptide into the crustaceans, thereby alters the sexual characteristics of the crustaceans. In one embodiment, the crustacean is a shrimp or lobster.

In another embodiment, the present invention provides a method of obtaining a monosex culture of female crustacean by introducing a recombinant IAG peptide into normal females and crossing the resulted offspring with normal females, thereby produces a monosex culture of female crustacean.

### BRIEF DESCRIPTION OF THE DRAWINGS

FIG. 1 shows a schematic diagram which shows the production of a monosex culture of female shrimp by introducing a recombinant IAG peptide into female shrimps and selected mating.

### DETAILED DESCRIPTION OF THE INVENTION

Crustaceans such as shrimps and lobsters have two sex chromosomes W and Z. Female gamete bears either W or Z chromosome while the male gamete contains only the Z chromosome. Therefore, female gamete determines the sex of an individual, i.e. an individual having a genotype of WW or ZW would be a female while a genotype of ZZ would give a male.

The present invention provides a nucleic acid molecule and method for manipulating sex, reversing sex or changing sex ratio in crustaceans by moderating the expression of crustacean androgenic gland peptide in crustaceans. In another embodiment, the present invention provides a nucleic acid molecule and method for producing a monosex culture of crustacean.

In one embodiment, the nucleic acid molecule of the present invention is a double-stranded RNA (dsRNA) corresponding to the DNA sequence of the insulin-like androgenic gland peptides (IAG) of crustacean.

In one embodiment, the nucleic acid molecule is a double-stranded RNA (dsRNA) corresponding to the DNA sequence of the insulin-like androgenic gland peptides (IAG) of shrimp.

In one embodiment, the nucleic acid molecule is a double-stranded RNA (dsRNA) corresponding to the DNA sequence of the insulin-like androgenic gland peptides (IAG) of Penaeus monodon of SEQ ID NO:1.

In one embodiment, the nucleic acid molecule is a double-stranded RNA (dsRNA) corresponding to the DNA sequence of the insulin-like androgenic gland peptides (IAG) of Litopenaeus vannamei of SEQ ID NO:2.

In one embodiment, the nucleic acid molecule is a double-stranded RNA (dsRNA) corresponding to the DNA sequence of the insulin-like androgenic gland peptides (IAG) of Fenneropenaeus chinensis of SEQ ID NO:3.

In one embodiment, the nucleic acid molecule is a double-stranded RNA (dsRNA) corresponding to the DNA sequence of the insulin-like androgenic gland peptides (IAG) of lobster.

In one embodiment, the nucleic acid molecule is a double-stranded RNA (dsRNA) corresponding to the DNA sequence of the insulin-like androgenic gland peptides (IAG) of other crustacean species.

The sequences of dsRNA for IAG gene of Penaeus monodon, Litopenaeus vannamei and Fenneropenaeus chinensis are:

Penaeus monodon
(SEQ ID NO: 1)
ATGAACCAACTCGCTGCCTCACGCACCTACGGCCTCGGCATGG
CCACTCAGCTGCTTGTGGGAATGCTGATGGTCCTCTCCCTGAC
TTCGGCGTCAAGCTGCTACAACGTCACGGGGATTCCTGTTGAC
TTCGACTGCGGTGACATCGGCGATACCATGAGTCACATCTGCA
AGACGTTTCCCACAGCCCGGCCTCACTCGCGAATGCCTTCTCT
TTCCCTTACAGTGTCCAGGTCAGCTGATACCGACGACCTCTGG
CAGGACACGAGAGCAGGTCAGACCACGCCCATTGACCTGCTTC
CTCGCCAGTACCGCCTGCATCCCCGGGCCTTGAATCCGATGCG
ATATTTCGAAATGGACCTGATAAGAGACCACCTGGTGAGCCCC
GAAGCCGCGCACGCCCTCGTCAAGACATCCAGGAGGCGCGCGA
AGAGATCCTACAACGTGCAGGATGAGTGCTGCAACCACGTGAG
CCAGCGGACGTGTGTGGCGGAGGAGATTCTGGAGTATTGCGAG
GACCCGTACTTCTAA
Litopenaeus vannamei
(SEQ ID NO: 2)
atctgcaaga cgtttcccac ggccaggccc cacgtgagag
tgtcaaggtc agccgatacc gacgacctct ggcaggacgc
gggggcaggt cagacaacgc cccctgacct gctcccccgc
cggcaccgcc tccaccccag ggccctgaat ccaacgtgga
atctcgaaag ggacctgatc agagacatcc tagtgagccc
cgaagccgcg cacgccctcg tcaggacgcc ccggagccgc
gcgaagaggt cctacaacgt gcaggacgag tgctgcaacc
acgtgagcca gcggatgtgt gtggcggagg agatcctgga
gtattgcgag gacccgtac
Fenneropenaeus chinensis
(SEQ ID NO: 3)
atgatccctc tcactgcctc gcgcccccac ggcctcggca
tacccactca actgctcgtg ggcgtgctgg tggtcctttc
cctgacttcg acgtcgagtg gctacaacgt caccgggatt
cctgttgact tcgactgcgg ggacatcggc gacaccatga
gtcagatctg caagacgttc cccacggcca ggccttacgc
gcgagtgtca aggtcagctg ataccgacga cctctggcat
gacacgggag cagatcagac cacgcccctt gacctgctca
gtcgccagta tcgcctccac tgaatccgat gcgatatctc
gaaagggacc tgacaaagca catcctggtg agccgcgaag
ccgcgcacgc cctcgtcaag acatccggga gtcgcgtgaa
gagatcctac aacgtgcagg acgagtgctg caaccacgtg
agccagcgga cgtgtgtggc ggaggagatt ctggagtatt
gcgaggaccc ggtaccttag

In one embodiment, the crustacean androgenic gland peptide is moderated for producing a monosex culture of female shrimp.

In another embodiment, the crustacean androgenic gland peptide is moderated for producing a monosex culture of female Penaeus monodon.

In one embodiment, the crustacean androgenic gland peptide is moderated for producing a monosex culture of female Litopenaeus vannamei.

In one embodiment, the crustacean androgenic gland peptide is moderated for producing a monosex culture of female Fenneropenaeus chinensis.

In one embodiment, the crustacean androgenic gland peptide is moderated for producing a monosex culture of female lobster.

In one embodiment, the crustacean androgenic gland peptide is moderated for producing a monosex culture of other female crustacean species.

In one embodiment, the present invention provides a method of producing a monosex culture of female crustacean comprising the step of injecting a dsRNA for the gene of JAG peptide into a male crustacean. In one embodiment, the dsRNA to be injected is a dsRNA corresponding to the DNA sequence of the gene of IAG of male shrimp or lobster, thereby producing a monosex culture of female shrimp or lobster.

As discussed above, current methods which use dsRNA for knocking down IAG gene are ineffective because the dsRNA is not injected at the time IAG gene is expressed. It is noted that expression of IAG is specific to certain molt stages. Injection of dsRNA at an inappropriate molt stage may induce no sex reversal or result in delay responses. The present method selects an appropriate molt stage for dsRNA injection such that crustacean would be more responsive to the dsRNA, and thereby enhances the efficiency in moderation of IAG and sex reversal.

In one embodiment, the dsRNA for IAG is introduced into the shrimps once the shrimps have molted. In another embodiment, the dsRNA for IAG is introduced into the shrimps for many molt cycles that result in gradual change of sex.

In one embodiment, the time to inject the dsRNA is when the shrimp or lobster is young or sexually undifferentiated.

In one embodiment, dsRNA is injected into male shrimp when the shrimp is about 2.5-3.0 cm long.

In one embodiment, the shrimp is injected with the dsRNA for once. In another embodiment, after the first injection, the shrimp is injected with the dsRNA for a second time when it molts again.

In one embodiment, the present invention provides a method of producing a monosex culture of female crustacean comprising the step of injecting a dsRNA for the gene of IAG into a female crustacean that had acquired a spermatophore (a sperm sac) after mating with a male.

In one embodiment, the female is artificially inseminated with a spermatophore (a sperm sac). The dsRNA for IAG gene can be introduced into the female together with the sperm sac and induces silencing of the IAG gene in the female. When the female spawns, the eggs (which contain either a Z or W chromosome) will be fertilized by the sperm (with Z chromosome). The eggs with W chromosome that are fertilized by the Z sperm will become a normal female (ZW). The eggs with Z chromosome that are fertilized by the Z sperm will turn into a normal male (ZZ). However, since the gene of IAG would be knocked down by the dsRNA, these normal male will turn into a “neo-female”. Accordingly, a 100% female population can be obtained from one single cross of the animal.

In one embodiment, the method of producing a monosex culture comprises the step of artificially inseminating female shrimps or lobsters with a spermatophore (a sperm sac). The sperm sac can be introduced into the shrimps (e.g. P. monodon and F. chinensis) or lobsters when the females have molted.

In another embodiment, the sperm sac can be introduced into the shrimps (e.g. L. vannamei) when the females is about to spawn.

In one embodiment, the dsRNA for IAG gene can be introduced together with the sperm sac into the sperm receptacle by prior mixing the dsRNA solution with the sperm sac ejected from the male. The sperm sac, together with the dsRNA, will remain in the female for silencing the IAG gene. In one embodiment, the female is injected with the sperm sac and dsRNA 2 days before she spawns her egg.

In one embodiment, the female injected with the sperm sac and dsRNA for IAG gene releases the sperm when she ovulates or spawns her egg, leading to the fertilization of the eggs. In one embodiment, the eggs with W chromosome are fertilized by the Z sperm and become a normal female (ZW). In another embodiment, the eggs with Z chromosome are fertilized by the Z sperm and turn into a “neofemale” (ZZ) under the silencing effect of dsRNA for IAG.

In one embodiment, dsRNA for IAG is injected into female that have mated with the male naturally.

In another embodiment, dsRNA for IAG is injected into the thylecum of the female shrimp that have been mated.

In one embodiment, the method of producing a monosex culture of female shrimp or lobster comprises the following steps:
a) obtaining sperm sac from the male shrimp or lobster;
b) mixing dsRNA for IAG with the sperm sac;
c) performing artificial insemination to insert the mixture obtained from step b) into a female shrimp or lobster; and
d) culturing the female shrimp or lobster obtained from step c) till ovulation and fertilization occur, thereby obtaining a monosex culture of female shrimp or lobster.

In one embodiment, the sex of the shrimps or lobsters is differentiated using a sex marker after being injected with the dsRNA for IAG.

In one embodiment, the sex of the shrimps is differentiated using a sex marker AFLP after being injected with the dsRNA for IAG.

In one embodiment, the present invention provides a method of obtaining a monosex culture of female crustacean by introducing a recombinant or purified IAG peptide into a normal female and crossing the resulted offspring with a normal male, thereby produces a monosex culture of female crustacean.

In one embodiment, the recombinant or purified IAG peptide from shrimp or lobster is introduced into the female shrimp or lobster.

In one embodiment, the recombinant IAG peptide described herein is expressed in E. coli. In another embodiment, the recombinant IAG peptide described herein is expressed using other appropriate expression system.

In one embodiment, the IAG peptide is the IAG of Penaeus monodon encoded by the DNA sequence of SEQ ID NO:1. In another embodiment, the IAG peptide comprises the amino acid sequence of insulin-like androgenic gland peptide (IAG) of Penaeus monodon of SEQ ID NO:4.

In one embodiment, the IAG peptide is the IAG of Litopenaeus vannamei encoded by the DNA sequence of SEQ ID NO:2. In another embodiment, the recombinant IAG peptide comprises the amino acid sequence of insulin-like androgenic gland peptide (IAG) of Litopenaeus vannamei of SEQ ID NO:5.

In one embodiment, the IAG peptide is the IAG of Fenneropenaeus chinensis encoded by the DNA sequence of SEQ ID NO:3. In another embodiment, the recombinant IAG peptide comprises the amino acid sequence of insulin-like androgenic gland peptide (IAG) of Fenneropenaeus chinensis of SEQ ID NO:6.

In one embodiment, the recombinant IAG peptide comprises the amino acid sequence of insulin-like androgenic gland peptide (IAG) of Macrobrachium rosenbergii (Fresh water shrimp) of SEQ ID NO:7.

In one embodiment, the recombinant IAG peptide comprises the amino acid sequence of insulin-like androgenic gland peptide (IAG) of Jasus edwardsii (Spiny lobster) of SEQ ID NO:8.

Below are the amino acid sequences of IAG gene that may be used in the present invention:

Penaeus monodon (Tiger shrimp, Genbank #GU208677)
(SEQ ID NO: 4)
MNQLAASRTY GLGMATQLLV GMLMVLSLTS ASSCYNVTGI
PVDFDCGDIG DTMSHICKTF PTARPHSRMP SLSLTVSRSA
DTDDLWQDTR AGQTTPIDLL PRQYRLHPRA LNPMRYFEMD
LIRDHLVSPE AAHALVKTSR RRAKRSYNVQ DECCNHVSQR
TCVAEEILEY CEDPYF
Litopenaeus vannamei
(SEQ ID NO: 5)
ICKTFPTARP HVRVSRSADT DDLWQDAGAG QTTPPDLLPR
RHRLHPRALN PTWNLERDLI RDILVSPEAA HALVRTPRSR
AKRSYNVQDE CCNHVSQRMC VAEEILEYCE DPY
Fenneropenaeus chinensis
(SEQ ID NO: 6)
MIPLTASRPH GLGIPTQLLV GVLVVLSLTS TSSGYNVTGI
PVDFDCGDIG DTMSQICKTF PTARPYARVS RSADTDDLWH
DTGADQTTPL DLLSRQYRLH PRALNPMRYL ERDLTKHILV
SREAAHALVK TSGSRVKRSY NVQDECCNHV SQRTCVAEEI
LEYCEDPVP
Macrobrachium rosenbergii (Fresh water shrimp)
(SEQ ID NO: 7)
MGYWNAEIKC VLFCSLVASL LPQPSSSYEI ECLSVDFDCG
DITNTLASVC LRHNNYINPG PTYVSKERRS ADIYTVPSTK
SPSLAHPRAT HLTMADEETQ KVSKVEEEIQ HMTLSREEAN
NMLHSKRRFR RDSVRRSPRE ECCNNASFRR CNFEEVAEYC
IELRPGVNTC SSR
Jasus edwardsii (Spiny lobster, Genbank #AIM55892)
(SEQ ID NO: 8)
MLAPILLKLV VIAGMRPRPV VSYRVEGLSV DFQCGELEDS
INSICAEHRE KRSVNTAAVG ASRDGGTDPS RRPYRHPRAI
QMVLSHPTTL AGAVQQGAGA VEGVRVTRQA ALSYVKSRIQ
DTNETNLQDE CCPVPLVRHC EMEEILEYCY LMEG

In one embodiment, the method of obtaining a monosex culture of female shrimp or lobster comprising the following steps:
a) Selecting female shrimp or lobster of genotype (ZW) during early stage of sexual development (i.e. 2-3 cm) at intermolt stage;
b) Injecting the selected female with a recombinant IAG peptide;
c) Injecting the selected female with a recombinant IAG peptide for a second time when the female reaches the intermolt stage of the next molting cycle;
d) Culturing the selected female until the female develops a male sexual characteristic(s), thereby obtaining the neomale population;
e) Mating the neomale with a normal female shrimp or lobster of genotype (ZW), thereby producing a population of shrimp or lobster with genotypes WW (female), ZW (female) or ZZ (male);
f) Screening the population of shrimp or lobster obtained from step e) for genotype WW using appropriate DNA markers; and
g) Mating the shrimp or lobster having a genotype WW obtained from step f) with a normal male (ZZ) shrimp or lobster, thereby obtaining a monosex culture of female shrimp or lobster.

In contrast to current methods, the present method introduces IAG protein into the shrimps at the intermolt stage. It is observed that a higher population of shrimps was responsive to IAG protein treatment at intermolt stage and underwent sex reversal in comparison with treatments performed in other stages.

In one embodiment, the IAG peptide is introduced into the shrimps or lobsters for many molt cycles that result in gradual change of sex.

The present invention provides a method of producing a monosex culture of crustacean, comprising the steps of
a) obtaining a sperm sac from a male crustacean;
b) mixing the sperm sac with a double-stranded RNA (dsRNA) comprising a nucleotide sequence of crustacean insulin-like androgenic gland peptide (IAG), thereby obtaining a mixture of the sperm sac and the dsRNA;
c) contacting the mixture obtained from step b) with a female crustacean, wherein the eggs of the female crustacean are fertilized by the sperm sac in the mixture, thereby obtaining a plurality of fertilized eggs; and
d) culturing the fertilized eggs obtained from step c) under appropriate conditions to obtain a monosex culture of crustacean having the phenotype of a female crustacean.

In one embodiment, the crustacean is a shrimp or lobster.

In one embodiment, the crustacean is Penaeus monodon, Litopenaeus vannamei or Fenneropenaeus chinensis.

In one embodiment, the double-stranded RNA (dsRNA) comprises a nucleotide sequence selected from the group consisting of SEQ ID NOs:1-3.

In one embodiment, the crustacean in the monosex culture has homozygous sex chromosomes or heterozygous sex chromosomes.

The present invention provides a method of producing a monosex culture of crustacean, comprising the steps of
a) obtaining a female crustacean having a desired set of sex chromosomes;
b) introducing the female crustacean with a crustacean insulin-like androgenic gland (IAG) peptide;
c) introducing the female crustacean obtained from step b) with a crustacean IAG peptide for a second time;
d) culturing the female crustacean obtained from step c) until the female crustacean develops one or more male sexual characteristics, thereby obtaining a population of male crustacean having the phenotype of a female crustacean;
e) mating the population of male crustacean obtained from step d) with a female crustacean, thereby obtaining a first generation of crustacean;
f) selecting a female crustacean having desired set of sex chromosomes from the first generation of crustacean; and
g) mating the crustacean selected in step f) with a normal male crustacean, thereby obtaining a monosex culture of crustacean having the phenotype of a female crustacean.

In one embodiment, the crustacean is a shrimp or lobster.

In one embodiment, the crustacean is Penaeus monodon, Litopenaeus vannamei Fenneropenaeus chinensis, Macrobrachium rosenbergii or Jasus edwardsii.

In one embodiment, the crustacean insulin-like androgenic gland (IAG) peptide is encoded by a nucleotide sequence selected from the group consisting of SEQ ID NOs:1-3.

In one embodiment, the crustacean insulin-like androgenic gland (IAG) peptide is an isolated or recombinant protein comprising an amino acid sequence selected from the group consisting of SEQ ID NOs:4-8.

In one embodiment, the female crustacean obtained from step b) in above method is introduced with the crustacean insulin-like androgenic gland (IAG) peptide for a second time when the female crustacean reaches the intermolt stage of the next molting cycle.

In one embodiment, the crustacean insulin-like androgenic gland (IAG) peptide is introduced in combination with other chemicals, peptides, proteins, or reagents.

The present invention provides a method of producing a monosex culture of crustacean, comprising the step of injecting a double-stranded RNA (dsRNA) comprising the nucleotide sequence of the crustacean insulin-like androgenic gland (IAG) peptide into a male crustacean, thereby obtaining a culture of crustacean having the phenotype of a female crustacean.

In one embodiment, the crustacean is a shrimp or lobster.

In one embodiment, the crustacean is Penaeus monodon, Litopenaeus vannamei or Fenneropenaeus chinensis.

In one embodiment, the double-stranded RNA (dsRNA) comprises a nucleotide sequence selected from the group consisting of SEQ ID NOs:1-3.

This invention will be better understood by reference to the examples which follow. However, one skilled in the art will readily appreciate that the examples provided are merely for illustrative purposes and are not meant to limit the scope of the invention which is defined by the claims following thereafter.

Throughout this application, it is to be noted that the transitional term “comprising”, which is synonymous with “including”, “containing” or “characterized by”, is inclusive or open-ended, and does not exclude additional, un-recited elements or method steps.

### EXAMPLE 1

### Knock-Down IAG Gene Expression by RNA Interference

To produce an all female shrimp culture, a simple approach is to inject dsRNA for IAG gene into a male shrimp. In one embodiment, the best time to inject the dsRNA is when they are young, or sexually undifferentiated. For example, for P. monodon (largest female sometime reach 23 cm or 180 grams), one should inject them when they are about 2.5-3.0 cm long. Inject all the small shrimp and later determine if all of the shrimp are female. A deviation of the 1:1 ratio.

In another embodiment, juvenile shrimp of 2.5-3.0 cm is selected for sex identification. Twenty male shrimps is selected and injected with dsRNA for IAG (e.g. 1 μg/g of body weight). The control male shrimps are injected with PBS only. The two groups are cultured separately and the same injection procedure is repeated when they molt again. Most of the shrimps injected with the dsRNA should lose the secondary sexual characteristics (e.g. the appendix masculine of the swimming leg will disappear or reduce in size).

Sex markers can be used to differentiate the male from the female for younger shrimp. In one embodiment, the sex marker AFLP described in Staelens, 2008 can be used for Penaues monodon [12]. This method is useful for screening out the male for dsRNA injection when the animal is small.

### EXAMPLE 2

### Knock-Down IAG Gene Expression by RNA Interference using a Spermatophore

Shrimp can be artificially inseminated with a spermatophore (a sperm sac). The sperm sac is collected from the male shrimp. dsRNA for the IAG gene is mixed with the collected sperm sac. When the female molts and the exoskeleton is soft, the sperm sac with dsRNA is inserted into the female thylecum (sperm receptacle), preferably 1-2 days after she molted. The sperm sac, together with the dsRNA, will remain in the female body and silence the IAG gene. Ovary will continue to develop for the next 5-6 days. The female is injected with dsRNA for the IAG gene (e.g. 10 ug/g of body weight) 1 to 2 days before she spawns.

When the female spawns, the eggs (which contain either a Z or W chromosome) will be fertilized by the sperm (with Z chromosome). The eggs with W chromosome that are fertilized by the Z sperm will become a normal female (ZW). The eggs with Z chromosome that are fertilized by the Z sperm will turn into a normal male (ZZ). However, since the gene of IAG would be knocked down by the dsRNA, these normal male will turn into a “neofemale”.

dsRNA for IAG can also be injected into female shrimp that have mated with the male naturally. In another embodiment, dsRNA for IAG can be injected into the thylecum of the female that have been mated which requires lower dose of dsRNA.

### EXAMPLE 3

### Obtaining a Monosex Culture Using Recombinant IAG Peptide

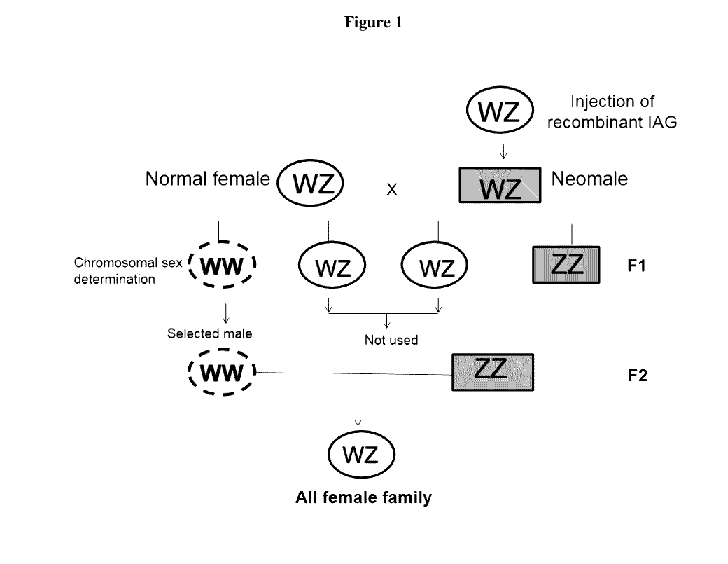

As illustrated in FIG. 1, a monosex culture of female crustacean such as shrimp or lobster can also be obtained by introducing a recombinant IAG peptide into the female crustacean.

Firstly, female crustacean such as shrimps (ZW) are selected during early stage of sexual development at intermolt stage. In one embodiment, shrimps at the early stage of 2-3 cm in total length are selected for injection. In another embodiment, shrimps at the early stage of 3-4 cm in total length are selected for injection.

The selected shrimps are then injected with a recombinant protein for IAG (insulin-like androgenic gland hormone). When the females reach the intermolt stage of the next molting cycle, the females are injected with a recombinant protein for IAG again. In one embodiment, the concentration of recombinant IAG protein used for injection is slightly higher than the physiological concentration of the IAG protein in the shrimps. In another embodiment, the concentration of recombinant IAG protein used for injection is at the range of 5-20 μg/g of body weight. Injection at an early stage would enhance the efficiency of sex reversal with a lower dose of recombinant IAG protein.

The treated females are then cultured until they have completely converted into a functional males (“neomales”) (ZW). This can be observed by the development of a male sex organ such as pestama with the spermatophore.

The “neomales” obtained is then mated with a normal female (ZW) to produce the F1 generation having a genotype of ZZ, ZW, ZW or WW. Accordingly, one-third of the F1 generation will be male (ZZ) and the remaining F1 generation will be female (ZW or WW).

The female progeny having the WW phenotype is desirable and selected using appropriate DNA sex marker. The obtained female progeny (WW) is then mated with a normal male (ZZ), and finally produce all-female progeny having a genotype of WZ.

### EXAMPLE 4

### Sexual Determination Using Sex Markers

Sex markers can be used to assess the progress of sex reversal in the treated organisms, or to differentiate male and female organisms. In one embodiment, the sex marker AFLP described in Staelens, 2008 can be used for Penaues monodon [12]. In another embodiment, the sex marker described in Jiang, 2013 can be used for Macrobrachium rosenbergii [13].

### REFERENCES

1. Sagi A (2013) Monosex culture of prawns through androgenic gene silencing. INFOFISH International 1, 22-24
2. Ventura T & Sagi A (2012) The insulin-like androgenic gland hormone in crustaceans: From a single gene silencing to a wide array of sexual manipulation-based biotechnologies. Biotechnol Adv 30(6), 1543-50.
3. Ohs C L, D'Abramo L R & Kelly A M (2006) Effect of dietary administration of 17α-methyltestosterone on the sex ratio of postlarval freshwater prawn, Macrobrachium rosenbergii, during the nursery stage of culture. JWorld Aquacult Soc 37, 328-33.
4. Ventura T, Aflalo E D, Weil S, Kashkush K & Sagi A (2007) Isolation and characterization of a female-specific DNA marker in the giant freshwater prawn Macrobrachium rosenbergii. Heredity 107, 456-61.
5. Aflalo E D, Hoang T T T, Nguyen V H, Lam Q, Nguyen D M, Trinh Q S, et al. (2006) A novel two-step procedure for mass production of all-male populations of the giant freshwater prawn Macrobrachium rosenbergii. Aquaculture 256, 468-78.
6. Ventura T,Manor R, Aflalo E D, Weil S, Rosen O & Sagi A (2012) Timing sexual differentiation: full functional sex reversal achieved in Macrobrachium rosenbergii through silencing of a single insulin-like gene. Biol Reprod 86(3), 90, 1-6.
7. Parnes S, Khalaila I, Hulata G & Sagi A (2003) Are intersex crayfish Cherax quadricarinatus (VonMartens) genetically females? Gen Res 82, 107-16.
8. Tri\ddot{n}o A T, Millamena O M & Keenan C (1999) Commercial evaluation of monosex pond culture of the mud crab Scylla species at three stocking densities in the Philippines. Aquaculture 174, 109-18.
9. Curtis M C & Jones C M (1995) Observations on monosex culture of redclaw crayfish Cherax quadricarinatus von Martens (Decapoda: Parastacidae) in earthen ponds. J World Aquacult Soc 26, 154-9.
10. Manor R, Segev R, Leibovitz M P, Aflalo E D & Sagi A (2002) Intensification of redclaw crayfish Cherax quadricarinatus culture I: Growout in a separate cell system. Aquacult Eng 26,263-76.
11. Sagi A, Ra'anan Z, Cohen D & Wax Y (1986) Production of Macrobrachium rosenbergii in monosex population: yield characteristics under intensive monoculture conditions in cages. Aquaculture 51,265-75.
12. Staelens J, Rombaut D, Vercauteren I, Argue B, Benzie J & Vuylsteke M (2008) High-density linkage maps and sex-linked markers for the black tiger shrimp (Penaeus monodon). Genetics 179(2), 917-25.
13. Jiang X H & Qiu GF (2013) Female-only sex-linked amplified fragment length polymorphism markers support ZW/ZZ sex determination in the giant freshwater prawn Macrobrachium rosenbergii. Anim Genet. 44(6):782-5.

What is claimed is:

1. A method of producing a monosex culture of crustacean, comprising the steps of
a) obtaining a sperm sac from a male crustacean;
b) mixing the sperm sac with a double-stranded RNA (dsRNA) comprising a nucleotide sequence of crustacean insulin-like androgenic gland peptide (IAG), thereby obtaining a mixture of the sperm sac and the dsRNA;
c) contacting the mixture obtained from step b) with a female crustacean, wherein the eggs of the female crustacean are fertilized by the sperm sac in the mixture, thereby obtaining a plurality of fertilized eggs; and
d) culturing the fertilized eggs obtained from step c) under appropriate conditions to obtain a monosex culture of crustacean having the phenotype of a female crustacean.

2. The method of claim 1, wherein the crustacean is a shrimp or lobster.

3. The method of claim 1, wherein the crustacean is Penaeus monodon, Litopenaeus vannamei or Fenneropenaeus chinensis.

4. The method of claim 1, wherein the double-stranded RNA (dsRNA) comprises a nucleotide sequence selected from the group consisting of SEQ ID NOs:1-3.

5. The method of claim 1, wherein crustacean in the monosex culture has homozygous sex chromosomes or heterozygous sex chromosomes.

6. A method of producing a monosex culture of crustacean, comprising the steps of
a) obtaining a female crustacean having a desired set of sex chromosomes;
b) introducing the female crustacean with a crustacean insulin-like androgenic gland (IAG) peptide;
c) introducing the female crustacean obtained from step b) with a crustacean TAG peptide for a second time;
d) culturing the female crustacean obtained from step c) until the female crustacean develops one or more male sexual characteristics, thereby obtaining a population of male crustacean having the phenotype of a female crustacean;
e) mating the population of male crustacean obtained from step d) with a female crustacean, thereby obtaining a first generation of crustacean;
f) selecting a female crustacean having desired set of sex chromosomes from the first generation of crustacean; and
g) mating the crustacean selected in step f) with a normal male crustacean, thereby obtaining a monosex culture of crustacean having the phenotype of a female crustacean.

7. The method of claim 6, wherein the crustacean is a shrimp or lobster.

8. The method of claim 6, wherein the crustacean is Penaeus monodon, Litopenaeus vannamei Fenneropenaeus chinensis, Macrobrachium rosenbergii or Jasus edwardsii.

9. The method of claim 6, wherein the crustacean insulin-like androgenic gland (IAG) peptide is encoded by a nucleotide sequence selected from the group consisting of SEQ ID NOs:1-3.

10. The method of claim 6, wherein the crustacean insulin-like androgenic gland (IAG) peptide is an isolated or recombinant protein comprising an amino acid sequence selected from the group consisting of SEQ ID NOs:4-8.

11. The method of claim 6, wherein the female crustacean obtained from step b) is introduced with the crustacean insulin-like androgenic gland (IAG) peptide for a second time when the female crustacean reaches the intermolt stage of the next molting cycle.

12. The method of claim 6, wherein the crustacean insulin-like androgenic gland (IAG) peptide is introduced in combination with other chemicals, peptides, proteins, or reagents.

13. A method of producing a monosex culture of crustacean, comprising the step of injecting a double-stranded RNA (dsRNA) comprising the nucleotide sequence of the crustacean insulin-like androgenic gland (IAG) peptide into a male crustacean, thereby obtaining a culture of crustacean having the phenotype of a female crustacean.

14. The method of claim 13, wherein the crustacean is a shrimp or lobster.

15. The method of claim 13, wherein the crustacean is Penaeus monodon, Litopenaeus vannamei or Fenneropenaeus chinensis.

16. The method of claim 13, wherein the double-stranded RNA (dsRNA) comprises a nucleotide sequence selected from the group consisting of SEQ ID NOs:1-3.
